(19) United States
(12) Patent Application Publication
(10) Pub. No.: US 2023/0023005 A1
(43) Pub. Date: Jan. 26, 2023

(54) IMPROVING THE PERFORMANCE OF PIGMENT-BASED DYES BY USING A PRETREAMENT AGENT

(71) Applicant: Henkel AG & Co. KGaA, Duesseldorf (DE)

(72) Inventors: Constanze KRUCK, Grevenbroich (DE); Sandra HILBIG, Bochum (DE); Melanie MOCH, Dormagen (DE); Susanne DICKHOF, Viersen (DE); Daniela KESSLER-BECKER, Leverkusen (DE)

(73) Assignee: Henkel AG & Co. KGaA, Duesseldorf (DE)

(21) Appl. No.: 17/780,437

(22) PCT Filed: Sep. 14, 2020
(86) PCT No.: PCT/EP2020/075595

§ 371 Date: May 26, 2022

(30) Foreign Application Priority Data
Nov. 26, 2019 (DE) 10 2019 218 232.1

(51) Int. Cl. A61K 8/46; A61Q 5/06; A61K 8/898

(52) U.S. Cl. CPC A61K 8/463; A61Q 5/065; A61K 8/898

(57) ABSTRACT

The subject of the present disclosure is a process for dyeing keratinous material, in particular human hair, comprising the following steps:
Application of a pretreatment agent (V) to the keratinous material, the pretreatment agent being in a water-comprising carrier
   (V-1) comprises at least one anionic surfactant, and
Application of a colorant (F) to the keratinous material, wherein the colorant is
   (F-1) at least one amino-functionalized silicone polymer and
   (F-2) comprises at least one pigment.

A second object of the present disclosure is a multi-component packaging unit which comprises the two colorants (V) and (F) in two separately packaged containers.

### CROSS-REFERENCE TO RELATED APPLICATION

This application is a U.S. National-Stage entry under 35 U.S.C. § 371 based on International Application No. PCT/EP2020/075595, filed Sep. 14, 2020, which was published under PCT Article 21(2) and which claims priority to German Application No. 102019218232.1, filed Nov. 26, 2019, which are all hereby incorporated in their entirety by reference.

### TECHNICAL FIELD

The subject of the present application is a process for dyeing keratinous material, in particular human hair, which comprises the application of at least two different agents (V) and (F). The agent (V) represents a pretreatment agent comprising at least one anionic surfactant in a water-comprising carrier. The colorant (F) comprises at least one amino-functionalized silicone polymer (F-1) and at least one pigment (F-2).

A second subject of this application is a multi-component packaging unit (kit-of-parts) for dyeing keratinous material, in particular human hair, which comprises the two agents (V) and (F) separately assembled in two different containers.

### BACKGROUND

Changing the shape and color of keratinous material, especially human hair, is a key area of modern cosmetics. To change the hair color, the expert knows various coloring systems depending on the coloring requirements. Oxidation dyes are usually used for permanent, intensive dyeings with good fastness properties and good grey coverage. Such colorants contain oxidation dye precursors, so-called developer components and coupler components, which, under the influence of oxidizing agents such as hydrogen peroxide, form the actual dyes among themselves. Oxidation dyes are exemplified by very long-lasting dyeing results.

When direct dyes are used, ready-made dyes diffuse from the colorant into the hair fiber. Compared to oxidative hair dyeing, the dyeings obtained with direct dyes have a shorter shelf life and quicker wash ability. Dyes with direct dyes usually remain on the hair for a period of between 5 and 20 washes.

The use of color pigments is known for short-term color changes on the hair and/or skin. Color pigments are understood to be insoluble, coloring substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

If the user wants particularly long-lasting dyeings, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair. A continuing challenge is therefore the search for alternative, high-performance dyeing processes. One viable alternative coloring system that has recently come increasingly into focus is based on the use of colored pigments.

Coloring with pigments offers several significant advantages. Since the pigments only attach themselves to the keratin materials, especially the hair fibers, from the outside, the damage associated with the dyeing process is particularly low. Furthermore, colorations that are no longer desired can be removed quickly and easily without leaving any residue, thus offering the user the possibility of returning to his original hair color immediately and without significant effort. Especially for those consumers who do not want to recolor their hair regularly, this coloring process is therefore particularly attractive.

In recent work, the problem of low durability of this staining system has been addressed. In this context, it was found that the wash fastness of the color results obtained with pigments could be improved by combining the pigments with certain amino-functionalized silicone polymers. In addition, by selecting particularly well-suited pigments and pigment concentrations on dark hair, it was possible to achieve a lighter color result, so that with this coloring system it was even possible to lighten hair, which until then had only been possible with oxidative hair treatment agents (bleaching or bleaching agents).

In addition to these many advantages, however, the pigment-based coloring system still has some disadvantages. Since both the pigments and the amino silicones that immobilize the pigments are deposited on the surface of the hair fiber, the achievement of a uniform color result is highly dependent on the surface structure of the hair to be colored. The work carried out in this context has shown that even substances still present on the surface of the hair at the time of dyeing can have a very strong effect on the quality of a subsequent dyeing result. For example, people who had previously treated their hair with a conditioner or styling product obtained colorations with low intensity and extremely uneven color fallout. Without being committed to this theory, it is suspected that various ingredients included in care and/or styling products can accumulate in certain areas of the hair and prevent the formation of uniform color films there in a subsequent coloring process.

The user wants intense colorations with good uniformity and high reproducibility. Accordingly, a coloring system whose color result depends on the type and amount of another cosmetic product that may have been applied previously is highly unattractive in the eyes of the user.

### BRIEF SUMMARY

Processes and Kits-of-parts for dyeing keratinous material are provided herein. In an embodiment, a process for dyeing keratinous material includes the following steps:

Applying a pretreatment agent (V) to the keratinous material, the pretreatment agent being disposed in a water-containing carrier, wherein the pretreatment agent (V) comprises:
(V-1) at least one anionic surfactant, and

Applying a colorant (F) to the keratinous material, wherein the colorant comprises
(F-1) at least one amino-functionalized silicone polymer and
(F-2) at least one pigment.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description. It is to be appreciated that all numerical values as provided herein, save for the actual examples, are approximate values with endpoints or particular values intended to be read as “about” or “approximately” the value as recited.

It was the task of the present application to find a pigment-based dyeing process with which intensive dyeings with good reproducibility can be achieved. The keratin materials or hair dyed with this process should provide the exact shade and intensity of coloration that the user expects after reading the relevant packaging information or instructions for use, irrespective of the type of hair and other cosmetic products previously used. The colorations should have a high degree of uniformity and leveling, even with the prior application of a care or styling product.

Surprisingly, it has now been found that the task can be excellently solved if keratinous materials, in particular hair, are dyed using a two-stage process in which at least two agents (V) and (F) are applied to the keratinous materials (keratinous fibers such as hair). Here, the agent (V) is a pretreatment agent comprising at least one anionic surfactant (V-1) in an aqueous or water-comprising carrier. The pretreatment agent (V) is applied to the keratin materials before the application of the actual dyeing agent (F), allowed to act and, if necessary, rinsed out again. Following the application of the pretreatment agent (V), staining is performed by applying the actual staining agent (F) to the keratin material. The colorant (F) is exemplified by its content of at least one amino-functionalized silicone polymer (F-1) and at least one pigment (F-2).

A first object of the present disclosure is a method for coloring keratinous material, in particular human hair, comprising the following steps:

Application of a pretreatment agent (V) to the keratinous material, the pretreatment agent being in a water-comprising carrier
(V-1) comprises at least one anionic surfactant, and

Application of a colorant (F) to the keratinous material, wherein the colorant is
(F-1) at least one amino-functionalized silicone polymer and
(F-2) comprises at least one pigment.

In the work leading to the present disclosure, it has been shown that particularly intense and uniform color results can be obtained on hair when the hair has been colored by successive application of the two agents (V) and (F). Surprisingly, the intensity and uniformity of the color result were not affected even with prior application of a conditioner or styling agent.

### Keratinic Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair.

### Coloring Agent

The term “coloring agent” is used in the context of this present disclosure for a coloring of the keratin material, in particular the hair, caused using pigments. In this coloring process, the pigments are deposited as coloring compounds in a particularly homogeneous, uniform and smooth film on the surface of the keratin material.

### Pretreatment Agent (V)

In the process as contemplated herein, the pretreatment agent (V) is applied to the keratinous materials, to the keratin fibers, prior to application of the colorant (F).

The pretreatment agent (V) comprises at least one anionic surfactant (V-1) in an aqueous or water-comprising carrier.

### Water Content in the Pretreatment Agent

The pretreatment agent (V) comprises the anionic surfactant(s) (V-1) in an aqueous or water-comprising carrier. Preferably, the pretreatment agent (V) has a high water content. It has been found that particularly well suited for use in the process as contemplated herein are those pretreatment agents (V) which—based on the total weight of the pretreatment agent (V)—contain 50.0 to 98.0 wt. %, preferably 60.0 to 90.0 wt. %, further preferably 70.0 to 90.0 wt. % and very particularly preferably 75.0 to 90.0 wt. % water.

In one embodiment, the pretreatment agent (V) as contemplated herein comprises—based on the total weight of the pretreatment agent (V)—50.0 to 98.0 wt. %, preferably 60.0 to 90.0 wt. %, further preferably 70.0 to 90.0 wt. % and most preferably 75.0 to 90.0 wt. % water.

### Anionic Surfactants (V-1) in the Pretreatment Agent

As an essential ingredient, the pretreatment agent as contemplated herein comprises at least one anionic surfactant (V-1).

The term surfactants (T) refer to surface-active substances that can form adsorption layers on surfaces and interfaces or aggregate in bulk phases to form micelle colloids or lyotropic mesophases. A distinction is made between anionic surfactants including a hydrophobic radical and a negatively charged hydrophilic head group, amphoteric or zwitterionic surfactants, which carry both a negative and a compensating positive charge, cationic surfactants, which have a positively charged hydrophilic group in addition to a hydrophobic radical, and nonionic surfactants, which have no charges but strong dipole moments and are strongly hydrated in aqueous solution.

Certain anionic surfactants (V-1) have been found to be particularly suitable for achieving an intensive, uniform and reproducible color result in the subsequent application of a colorant (F) as contemplated herein.

Suitable anionic surfactants (V-1) that can be used in the agents of the present disclosure include:

linear and branched fatty acids with 8 to 30 C atoms (Soaps),

Ethercarboxylic acids of the formula R—O—(CH₂—CH₂O)ₓ—CH₂—COOH, in which R is a linear or branched, saturated or unsaturated alkyl group having 8 to 30 C atoms and x=0 or 1 to 16

Acylsarcosides with 8 to 24 C atoms in the acyl group,

Acyltaurides with 8 to 24 C atoms in the acyl group,

Acyl isethionates with 8 to 24 C atoms in the acyl group,

Sulphosuccinic acid mono- and/or dialkyl esters with 8 to 24 C atoms in the alkyl group and sulphosuccinic acid mono-alkyl polyoxyethyl esters with 8 to 24 C atoms in the alkyl group and 1 to 6 oxyethyl groups,

Alpha-olefin sulfonates with 8 to 24 C atoms,

Alkyl sulfate and/or alkyl polyglycol ether sulfate salts of the formula R—(OCH₂—CH₂)ₓ—OSO₃— X+, in which R is a preferably linear or branched, saturated or unsaturated alkyl group having 8 to 30 carbon atoms, x=0 or 1 to 12 and X is an alkali metal or ammonium ion,

Sulfonates of unsaturated fatty acids with 8 to 24 C atoms and 1 to 6 double bonds,

Esters of tartaric acid and citric acid with alcohols, which are addition products of about 2-15 molecules of ethylene oxide and/or propylene oxide to fatty alcohols with 8 to 22 C atoms,

Alkyl and/or alkenyl ether phosphates of the formula,

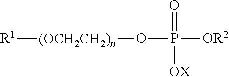

where R¹ is preferably an aliphatic hydrocarbon radical of 8 to 30 carbon atoms, R² is hydrogen, a radical (CH₂CH₂O)ₙR¹ or X, n is from 0 to 10 and X is hydrogen, an alkali metal or alkaline earth metal or NR³R⁴R⁵R⁶, where R³ to R⁶ are each independently of the others a C₁- to C₄-hydrocarbon radical.

Very particularly satisfactory results were obtained when at least one at least one anionic surfactant (V-1) of formula (T-1) was used in the pretreatment agent (V),

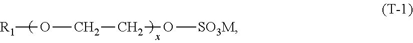

where

R¹ represents a linear or branched, saturated or unsaturated C₈-C₃₀ alkyl group,

x stands for an integer from 0 to 50, and

M is a hydrogen atom, ammonium (NH₄)+ or an equivalent of a monovalent or polyvalent cation.

In a very particularly preferred embodiment, the pretreatment agent (V) comprises (V-1) at least one anionic surfactant of the formula (T-1),

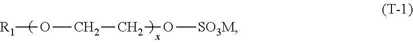

where

R₁ represents a linear or branched, saturated or unsaturated C₈-C₃₀ alkyl group,

x stands for an integer from 0 to 50, and

M is a hydrogen atom, ammonium (NH₄)+ or an equivalent of a monovalent or polyvalent cation.

The radical R1 represents the hydrophobic part of the anionic surfactant and stands for a linear or branched, saturated or unsaturated C₈-C₃₀ alkyl group. If the radical stands for an unsaturated C₈-C₃₀ alkyl group, then the alkyl group can be mono- or polyunsaturated.

Preferably, the radical R1 is a linear, saturated or unsaturated C₈-C₃₀ alkyl group-Very preferably, the radical R1 is a linear, saturated or unsaturated C₁₂-C₂₂ alkyl group. Explicitly quite particularly preferred, the radical R1 stands for an R for a linear, saturated or unsaturated C₁₂-C₁₈ alkyl group.

Examples of saturated, linear C₈-C₃₀ alkyl groups are the lauryl group, the myristyl group, the cetyl group, the stearyl group, and the behenyl group.

The index number x indicates the number of ethylene oxide groups included in the anionic surfactant. If x is 0, the anionic surfactant of formula (T-1) has no ethylene oxide units, in which case an alkyl sulfate or the salt of an alkyl sulfate is present.

Examples of the salts of alkyl sulfates are sodium lauryl sulfate and sodium myristyl sulfate.

Preferably, x represents an integer from 0 to 5, and particularly preferably, x represents an integer from 1 to 5.

The radical M stands for a hydrogen atom, for ammonium (NH₄)+ or for an equivalent of a monovalent or polyvalent cation.

If M stands for a hydrogen atom, then the anionic surfactant is present in the form of the protonated (and ethoxylated) sulfuric acid ester. In aqueous solution, the protonated form is in equilibrium with the deprotonated form, with the deprotonated form carrying an anionic charge. For this reason, the protonated compounds of formula (I) also fall into the group of anionic surfactants.

If M stands for ammonium (NH₄)+ or for an equivalent of a monovalent or polyvalent cation, then the anionic surfactant of formula (I) is present in the form of its salt. The presence of the corresponding equivalent of a monovalent or polyvalent cation here ensures the electroneutrality of the anion surfactant. Preferably, M represents a monovalent cation, in particular a sodium or potassium cation.

In a very particularly preferred embodiment, the pretreatment agent (V) comprises at least one anionic surfactant (V-1) of the formula (T-1), wherein

R1 is a linear, saturated or unsaturated C₁₂-C₁₈ alkyl group,

X is an integer from 0 to 5, preferably an integer from 1 to 5,

M represents a hydrogen atom or an alkali metal cation, preferably a sodium cation or a potassium cation.

A particularly preferred anionic surfactant of formula (I) can be obtained commercially, for example, under the trade name Texapon NSO BZ (BZ=preserved with benzoic acid) and the INCI designation Sodium Laureth Sulfate from BASF. This Sodium Laureth Sulfate has the CAS number 68891-38-3.

To ensure particularly good color results in the process as contemplated herein, the pretreatment agent preferably comprises the anionic surfactant(s) (V-1) in certain quantity ranges. Particularly intense and uniform colorations could be obtained if the pretreatment agent (V) included—based on the total weight of the pretreatment agent—one or more anionic surfactants (V-1) in a total amount of from 2.0 to 18.0 wt. %, preferably from 4.0 to 16.0 wt. %, more preferably from 6.0 to 14.0 wt. % and most preferably from 8.0 to 12.0 wt. %.

In a very particularly preferred embodiment, the pretreatment agent (V) comprises—based on the total weight of the pretreatment agent—one or more anionic surfactants (V-1) in a total amount of from 2.0 to 18.0 wt. %, preferably from 4.0 to 16.0 wt. %, more preferably from 6.0 to 14.0 wt. % and most preferably from 8.0 to 12.0 wt. %.

Zwitterionic and/or Amphoteric Surfactants in the Pretreatment Agent

The color effects achievable with the process as contemplated herein could be further improved in terms of intensity, uniformity and reproducibility if not only at least one anionic surfactant (V-1), but furthermore at least one zwitterionic and/or amphoteric surfactant were used in the pretreatment agent (V).

In a very particularly preferred embodiment, the pretreatment agent (V) is (V-2) comprises at least one zwitterionic and/or amphoteric surfactant.

Quite particularly suitable zwitterionic surfactants (V-2) may be selected from compounds of the following formulae (T-2), (T-3), (T-4) and/or (T-5),

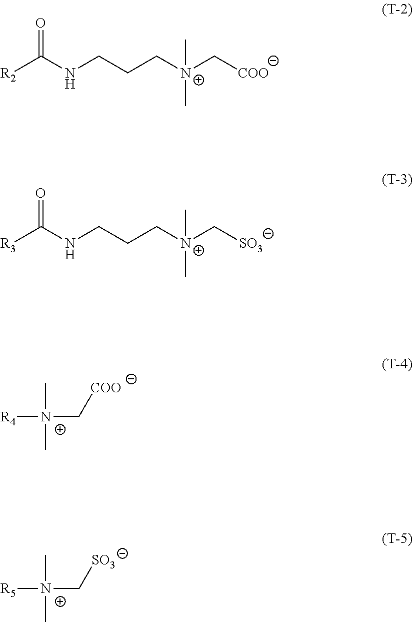

where

R2, R3, R4 and R5 each independently represent the hydrophobic radical of the surfactant. R2, R3, R4 and R5 independently represent a linear or branched, saturated or unsaturated C₈-C₃₀ alkyl group, preferably a linear, saturated or unsaturated C₁₂-C₁₈ alkyl group.

The zwitterionic surfactants of the formula (T-2), (T-3), (T-4) and/or (T-5) each have a cationic charge in the form of a quaternary nitrogen atom which, in addition to two methyl groups, carries the respective radical R and furthermore the radical comprising the acid function. This cationic charge is neutralized by the acid function, which may be a deprotonated carboxylic acid or sulfonic acid.

In a very particularly preferred embodiment, the pretreatment agent (V) is (V-2) comprises at least one zwitterionic surfactant selected from the surfactants of the formula (T-2), (T-3), (T-4) and/or (T-5),

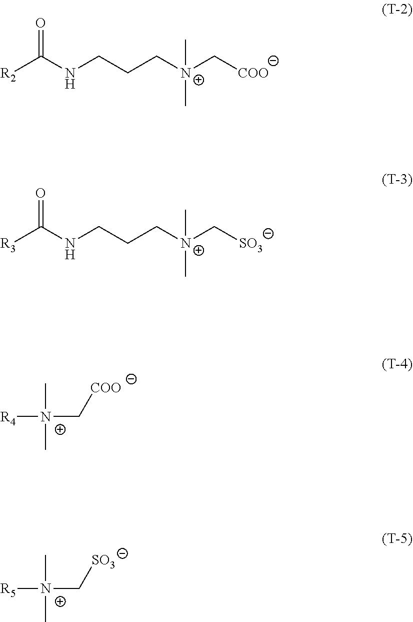

where
R2, R3, R4, R5 independently represent a linear or branched, saturated or unsaturated C₈-C₃₀ alkyl group, preferably a linear, saturated or unsaturated C₁₂-C₁₈ alkyl group.

The radical R2 represents a linear or branched, saturated or unsaturated C₈-C₃₀ alkyl group,

preferably for a linear, saturated or unsaturated C₁₁-C₂₁ alkyl group, very particularly
preferably for a linear, saturated or unsaturated C₁₁-C₁₇ alkyl group

The radical R3 represents a linear or branched, saturated or unsaturated C₈-C₃₀ alkyl group,

preferably for a linear, saturated or unsaturated C₁₁-C₂₁ alkyl group, very particularly
preferably for a linear, saturated or unsaturated C₁₁-C₁₇ alkyl group

The radical R4 represents a linear or branched, saturated or unsaturated C₈-C₃₀ alkyl group, preferably a linear, saturated or unsaturated C₁₂-C₁₈ alkyl group.

The radical R5 represents a linear or branched, saturated or unsaturated C₈-C₃₀ alkyl group, preferably a linear, saturated or unsaturated C₁₂-C₁₈ alkyl group.

Particularly suitable zwitterionic surfactants of formula (T-2) are alkylamidoalkyl betaines. Particularly suitable amphoteric surfactants include those known under the INCI designation cocamidopropyl betaine and the INCI name cocamidopropyl betaine.

Particularly suitable zwitterionic surfactants of formula (T-3) are, for example, C₁₂-C₁₄ alkyl dimethyl betaines, which can be obtained under the INCI designation Coco-Betaine in the form of the commercial product Genagen KB from the company Global Amines (formerly Clariant). Coco-Betaine has the CAS number 66455-29-6.

In particular, the zwitterionic surfactants of formula (T-2) and (T-4) have shown particularly good suitability for solving the problem as contemplated herein.

In the context of a further very particularly preferred embodiment, the pretreatment agent (V) comprises

(V-2) at least one zwitterionic surfactant selected from the surfactants of the formula (T-2) and/or (T-4).

To achieve particularly good dyeing results with the process as contemplated herein, the pretreatment agent preferably comprises the amphoteric or zwitterionic surfactants (V-2) in certain quantity ranges. Particularly intense and uniform colorations could be obtained if the pretreatment agent (V) included—based on the total weight of the pretreatment agent—one or more zwitterionic surfactants (V-2) in a total amount of 0.5 to 8.5 wt. %, preferably 1.0 to 7.5 wt. %, more preferably 1.5 to 6.5 wt. % and most preferably 2.0 to 4.5 wt. %.

In a very particularly preferred embodiment, the pretreatment agent (V) comprises —based on the total weight of the pretreatment agent—one or more zwitterionic and/or amphoteric surfactants (V-2) in a total amount of 0.5 to 8.5 wt. %, preferably 1.0 to 7.5 wt. %, more preferably 1.5 to 6.5 wt. % and most preferably 2.0 to 4.5 wt. %.

### Coloring Agent (F)

Following the application of the pretreatment agent (V), the colorant (F) is now applied to the keratin material in the process as contemplated herein. The colorant (F) comprises at least one amino-functionalized silicone polymer (F-1) and at least one pigment (F-2).

### Amino Functionalized Silicone Polymer (F-1) in Colorant (F)

As the first ingredient (F-1) essential to the present disclosure, the colorant (F) comprises at least one amino-functionalized silicone polymer. The amino-functionalized silicone polymer may alternatively be referred to as amino silicone or amodimethicone.

Silicone polymers are macromolecules with a molecular weight of at least 500 g/mol, preferably at least 1000 g/mol, more preferably at least 2500 g/mol, particularly preferably at least 5000 g/mol, which comprise repeating organic units.

The maximum molecular weight of the silicone polymer depends on the degree of polymerization (number of polymerized monomers) and the batch size and is partly determined by the polymerization method. For the purposes of the present disclosure, it is preferred if the maximum molecular weight of the silicone polymer is not more than 10⁷ g/mol, preferably not more than 10⁶ g/mol, and particularly preferably not more than 10⁵ g/mol.

The silicone polymers comprise many Si—O repeating units, and the Si atoms may carry organic radicals such as alkyl groups or substituted alkyl groups. Alternatively, a silicone polymer is therefore also referred to as polydimethylsiloxane.

Corresponding to the high molecular weight of silicone polymers, these are based on more than 10 Si—O repeat units, preferably more than 50 Si—O repeat units, and more preferably more than 100 Si—O repeat units, most preferably more than 500 Si—O repeat units.

An amino-functionalized silicone polymer is understood to be a functionalized silicone that carries at least one structural unit with an amino group. Preferably, the amino-functionalized silicone polymer carries multiple structural units, each having at least one amino group. An amino group is understood to mean a primary amino group, a secondary amino group and a tertiary amino group. All these amino groups can be protonated in the acidic environment and are then present in their cationic form.

In principle, beneficial effects could be obtained with amino-functionalized silicone polymers (F-1) if they carry at least one primary, at least one secondary and/or at least one tertiary amino group. However, dyeings with the best wash fastness were observed when an amino-functionalized silicone polymer (F-1) comprising at least one secondary amino group was used in the dye (F).

In a very particularly preferred embodiment, the colorant (F) comprises at least one amino-functionalized silicone polymer (F-1) having at least one secondary amino group.

The secondary amino group(s) may be located at various positions on the amino-functionalized silicone polymer. Particularly beneficial effects were found when an amino-functionalized silicone polymer (F-1) was used that has at least one, preferably several, structural units of the formula (Si amino).

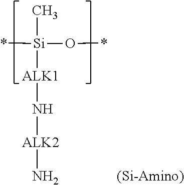

In the structural units of the formula (Si-Amino), the abbreviations ALK1 and ALK2 independently represent a linear or branched, divalent C₁-C₂₀ alkylene group.

In another very particularly preferred embodiment, the colorant (F) comprises at least one amino-functionalized silicone polymer (F-1) comprising at least one structural unit of the formula (Si-Amino),

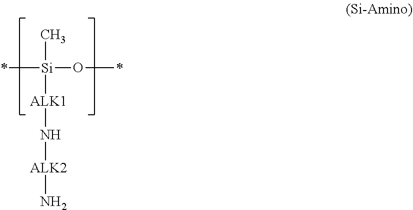

where
ALK1 and ALK2 independently represent a linear or branched C₁-C₂₀ divalent alkylene group.

The positions marked with an asterisk (*) indicate the bond to further structural units of the silicone polymer. For example, the silicon atom adjacent to the star may be bonded to another oxygen atom, and the oxygen atom adjacent to the star may be bonded to another silicon atom or even to a C₁-C₆ alkyl group.

A bivalent C₁-C₂₀ alkylene group can alternatively be referred to as a divalent or divalent C₁-C₂₀ alkylene group, by which is meant that each ALK1 or AK2 grouping can form two bonds.

In the case of ALK1, one bond occurs from the silicon atom to the ALK1 grouping, and the second bond is between ALK1 and the secondary amino group.

In the case of ALK2, one bond is from the secondary amino group to the ALK2 grouping, and the second bond is between ALK2 and the primary amino group.

Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In another particularly preferred embodiment, the structural units of the formula (Si amino) represent repeat units in the amino-functionalized silicone polymer (F-1), so that the silicone polymer comprises multiple structural units of the formula (Si amino).

Particularly well-suited amino-functionalized silicone polymers (F-1) with at least one secondary amino group are listed below.

Dyeings with the best wash fastnesses could be obtained if in the process as contemplated herein at least one dyeing agent (F) was applied to the keratinous material which comprises at least one amino-functionalized silicone polymer (F-1) comprising structural units of the formula (Si-I) and of the formula (Si-II)

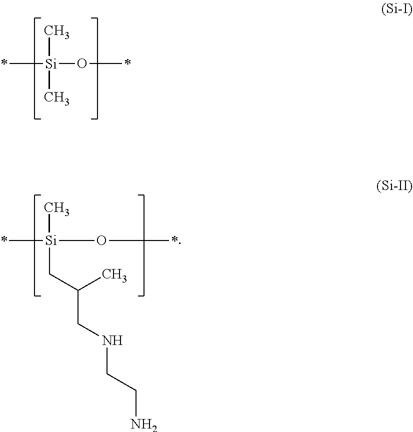

In a further explicitly quite particularly preferred embodiment, the colorant (F) comprises at least one amino-functionalized silicone polymer (F-1) which comprises structural units of the formula (Si-I) and of the formula (Si-II)

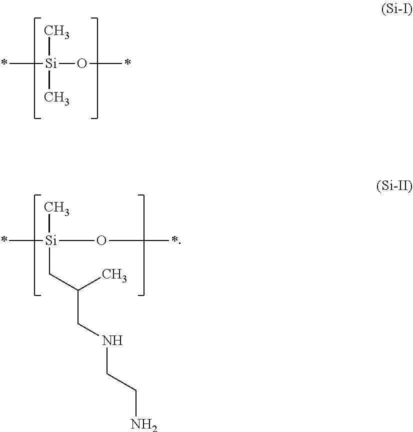

A corresponding amino functionalized silicone polymer with the structural units (Si-I) and (Si-II) is, for example, the commercial product DC 2-8566 or Dowsil 2-8566 Amino Fluid, which is commercially distributed by the Dow Chemical Company and bears the designation “Siloxanes and Silicones, 3-[(2-aminoethyl)amino]-2-methylpropyl Me, Di-Me-Siloxane” and the CAS number 106842-44-8.

In the context of a further preferred embodiment, a colorant (F) is applied to the keratinous material, the colorant (F) comprising at least one amino-functional silicone polymer (F-1) of the formula of formula (Si-III),

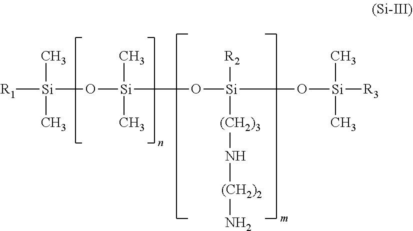

where
m and n mean numbers chosen so that the sum (n+m) is in the range 1 to 1000,
n is a number in the range 0 to 999 and m is a number in the range 1 to 1000,
R1, R2 and R3, which are the same or different, denote a hydroxy group or a C1-4 alkoxy group,
wherein at least one of R1 to R3 represents a hydroxy group;

Other processes preferred as contemplated herein include applying a colorant (F) to the keratinous material, the colorant (F) comprising at least amino-functional silicone polymer (F-1) of the formula of formula (Si-IV),

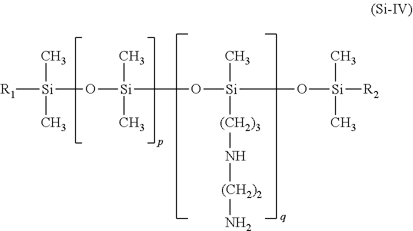

located in the
p and q mean numbers chosen so that the sum (p+q) is in the range 1 to 1000,
p is a number in the range 0 to 999 and q is a number in the range 1 to 1000,
R1 and R2, which are different, denote a hydroxy group or a C1-4 alkoxy group, at least one of R1 to R2 denoting a hydroxy group.

The silicones of the formulas (Si-III) and (Si-IV) differ in the grouping at the Si atom, which carries the nitrogen-comprising group: In formula (Si-III), R2 represents a hydroxy group or a C1-4 alkoxy group, while the radical in formula (Si-IV) is a methyl group. The individual Si groupings, which are marked with the indices m and n or p and q, do not have to be present as blocks; rather, the individual units can also be present in a statistically distributed manner, i.e. in the formulas (Si-III) and (Si-IV), not every R1-Si(CH₃)₂ group is necessarily bonded to an —[O—Si(CH₃)₂] grouping.

Processes as contemplated herein in which a colorant (F) comprising at least one amino-functional silicone polymer (F-1) of formula (Si-V) is applied to the keratin fibers have also proved to be particularly effective with respect to the desired effects.

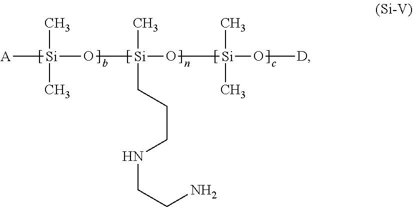

located in the
A represents a group —OH, —O—Si(CH₃)₃, —O—Si(CH₃)₂OH, —O—Si(CH₃)₂OCH₃,
D represents a group —H, —Si(CH₃)₃, —Si(CH₃)₂OH, —Si(CH₃)₂OCH₃,
b, n and c stand for integers between 0 and 1000, with the specifications
n>0 andb+c>0
at least one of the conditions A=—OH or D=—H is fulfilled.

In the above formula (Si-V), the individual siloxane units are statistically distributed with the indices b, c and n, i.e., they do not necessarily have to be block copolymers.

The colorant (F) may further comprise one or more different amino-functionalized silicone polymers represented by the formula (Si-VI)

M(RₐQ_{b}SiO_{(4-a-b)/2)x}(R_{c}SiO_{(4-c)/2)y}M (Si-VI)

in which formula above R is a hydrocarbon or a hydrocarbon radical having from 1 to about 6 carbon atoms, Q is a polar radical of the general formula —R¹HZ wherein R¹ is a divalent linking group bonded to hydrogen and the radical Z composed of carbon and hydrogen atoms, carbon, hydrogen and oxygen atoms, or carbon, hydrogen and nitrogen atoms, and Z is an organic amino functional radical comprising at least one amino functional group; “a” takes values ranging from about 0 to about 2, “b” takes values ranging from about 1 to about 3, “a”+“b” is less than or equal to 3, and “c” is a number ranging from about 1 to about 3, and x is a number ranging from 1 to about 2.000, preferably from about 3 to about 50 and most preferably from about 3 to about 25, and y is a number in the range of from about 20 to about 10,000, preferably from about 125 to about 10,000 and most preferably from about 150 to about 1,000, and M is a suitable silicone end group as known in the prior art, preferably trimethylsiloxy. Non-limiting examples of radicals represented by R include alkyl radicals, such as methyl, ethyl, propyl, isopropyl, isopropyl, butyl, isobutyl, amyl, isoamyl, hexyl, isohexyl and the like; alkenyl radicals, such as vinyl, halovinyl, alkylvinyl, allyl, haloallyl, alkylallyl; cycloalkyl radicals, such as cyclobutyl, cyclopentyl, cyclohexyl and the like; phenyl radicals, benzyl radicals, halohydrocarbon radicals, such as 3- chloropropyl, 4-bromobutyl, 3,3,3-trifluoropropyl, chlorocyclohexyl, bromophenyl, chlorophenyl and the like, and sulfur-comprising radicals, such as mercaptoethyl, mercaptopropyl, mercaptohexyl, mercaptophenyl and the like; preferably R is an alkyl radical comprising from 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂-, —CH₂CH₂OCH₂—, —OCH₂CH₂—, —OCH₂CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—.
Z is an organic amino functional radical comprising at least one amino functional group. One formula for Z is NH(CH₂)_{z}NH₂, where z is 1 or more. Another formula for Z is —NH(CH₂)_{z}(CH₂)_{zz}NH, wherein both z and zz are independently 1 or more, this structure comprising diamino ring structures, such as piperazinyl. Z is most preferably an —NHCH₂CH₂NH₂ radical. Another formula for Z is —N(CH₂)_{z}(CH₂)_{zz}NX₂ or —NX₂, wherein each X of X₂ is independently selected from the group of hydrogen and alkyl groups having 1 to 12 carbon atoms, and zz is 0.
Q is most preferably a polar, amine-functional radical of the formula —CH₂CH₂CH₂NHCH₂CH₂NH₂. In the formulas, “a” takes values ranging from about 0 to about 2, “b” takes values ranging from about 2 to about 3, “a”+“b” is less than or equal to 3, and “c” is a number ranging from about 1 to about 3. The molar ratio of RₐQ_{b}SiO_{(4-a-b>/2} units to R_{c}SiO_{(4-c)/2} units is in the range of about 1: 2 to 1: 65, preferably from about 1: 5 to about 1: 65 and most preferably by about 1: 15 to about 1: 20. If one or more silicones of the above formula are used, then the various variable substituents in the above formula may be different for the various silicone components present in the silicone mixture.

In a particularly preferred embodiment, a colorant (F) is applied to the keratinous material, wherein the colorant (F) is an amino-functional silicone polymer of formula (Si-VII)

R′ₐG₃₋ₐ-Si(OSiG₂)ₙ—(OSiG_{b}R′_{2-b})ₘ—O-SiG₃₋ₐ-R′ₐ (Si-VII),

wherein means:
G is-H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, especially 0;
b stands for a number between 0 and 1, especially 1,
m and n are numbers whose sum (m+n) is between 1 and 2000, preferably between 50 and 150, where n preferably assumes values from 0 to 1999 and from 49 to 149 and m preferably assumes values from 1 to 2000, from 1 to 10,
R′ is a monovalent radical selected from
   -Q-N(R″)—CH₂—CH₂—N(R″)₂
   -Q-N(R″)₂
   -Q-N⁺(R″)₃A-
   -Q-N⁺H(R″)₂A-
   -Q-N⁺H₂(R″)A-
   -Q-N(R″)—CH₂—CH₂—N+R″H₂A-,
      where each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
      R″ represents identical or different radicals selected from the group of —H, -phenyl, -benzyl, —CH₂—CH(CH₃)Ph, the C₁-20 alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion preferably selected from chloride, bromide, iodide or methosulfate.

In the context of a further preferred embodiment, a colorant (F) is applied to the keratinous material, the colorant (F) comprising at least one amino-functional silicone polymer (F-1) of formula (Si-VIIa),

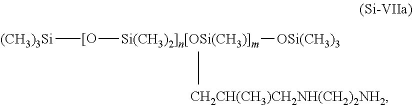

wherein m and n are numbers whose sum (m+n) is between 1 and 2000, preferably between 50 and 150, n preferably assuming values from 0 to 1999 and from 49 to 149, and m preferably assuming values from 1 to 2000, from 1 to 10.

According to the INCI declaration, these silicones are called trimethylsilylamodimethicones.

In the context of a further preferred embodiment, a colorant (F) is applied to the keratinous material, the colorant (F) comprising at least one amino-functional silicone polymer of the formula (Si-VIIb)

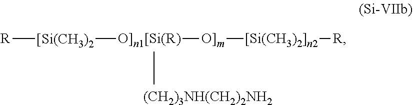

in which R represents —OH, —O—CH₃ or a —CH₃ group and m, n1 and n2 are numbers whose sum (m+n1+n2) is between 1 and 2000, preferably between 50 and 150, the sum (n1+n2) preferably assuming values from 0 to 1999 and from 49 to 149 and m preferably assuming values from 1 to 2000, from 1 to 10.

According to the INCI declaration, these amino-functionalized silicone polymers are called amodimethicones.

Regardless of which amino-functional silicones are used, colorants (F) as contemplated herein are preferred which contain an amino-functional silicone polymer whose amine number is above 0.25 meq/g, preferably above 0.3 meq/g and above 0.4 meq/g. The amine number represents the milliequivalents of amine per gram of the amino-functional silicone. It can be determined by titration and expressed in the unit mg KOH/g.

Furthermore, colorants (F) which included a special 4-morpholinomethyl-substituted silicone polymer (F-1) are also suitable for use in the process as contemplated herein. This amino-functionalized silicone polymer comprises structural units of the formulae (SI-VIII) and of the formula (Si-IX)

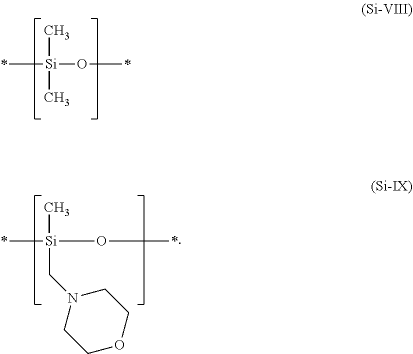

Corresponding 4-morpholinomethyl-substituted silicone polymers are described below.

A corresponding amino-functionalized silicone polymer is available under the name of

Amodimethicone/Morpholinomethyl Silsesquioxane Copolymer is known and commercially available from Wacker in the form of the raw material Belsil ADM 8301 E.
As a 4-morpholinomethyl-substituted silicone, for example, a silicone can be used which has structural units of the formulae (Si-VIII), (Si-IX) and (Si-X)

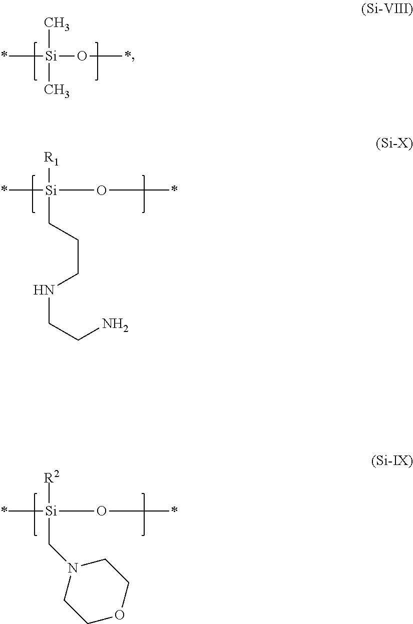

in which
R1 is —CH₃, —OH, —OCH₃, —O—CH₂CH₃, —O—CH₂CH₂CH₃, or —O—CH(CH₃)₂;
R2 is —CH₃, —OH, or —OCH₃.

Particularly preferred colorants (F) as contemplated herein contain at least one 4-morpholinomethyl-substituted silicone of the formula (Si-XI)

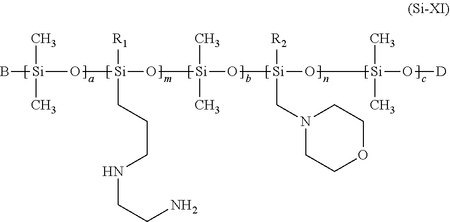

located in the
R1 is —CH₃, —OH, —OCH₃, —O—CH₂CH₃, —O—CH₂CH₂CH₃, or —O—CH(CH₃)₂;
R2 is —CH₃, —OH, or —OCH₃.
B represents a group —OH, —O—Si(CH₃)₃, —O—Si(CH₃)₂OH, —O—Si(CH₃)₂OCH₃,
D represents a group —H, —Si(CH₃)₃, —Si(CH₃)₂OH, —Si(CH₃)₂₀CH₃, a, b and c stand independently for integers between 0 and 1000, with the condition a+b+c>0
m and n independently of each other stand for integers between 1 and 1000

with the proviso that
at least one of the conditions B=—OH or D=—H is fulfilled,
the units a, b, c, m and n are distributed statistically or blockwise in the molecule.

Structural formula (Si-XI) is intended to illustrate that the siloxane groups n and m do not necessarily have to be directly bonded to a terminal grouping B or D, respectively. Rather, in preferred formulas (Si-VI) a>0 or b>0 and in particularly preferred formulas (Si-VI) a>0 and c>0, i.e., the terminal grouping B or D is preferably attached to a dimethylsiloxy grouping. Also, in formula (Si-VI), the siloxane units a, b, c, m and n are preferably statistically distributed.

The silicones used as contemplated herein represented by formula (Si-VI) can be trimethylsilyl-terminated (D or B=—Si(CH₃)₃), but they can also be dimethylsilylhydroxy-terminated on two sides or dimethylsilylhydroxy-terminated and dimethylsilylmethoxy-terminated on one side. Silicones particularly preferred in the context of the present disclosure are selected from silicones in which

B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH
to everyone. These silicones lead to exorbitant improvements in the hair properties of the hair treated with the agents of the present disclosure, and to a seriously improved protection in oxidative treatment.

It has been found to be particularly advantageous if the colorant as contemplated herein comprises the amino-functionalized silicone polymer(s) (F-1) in certain ranges of amounts. Particularly satisfactory results were obtained when the colorant—based on the total weight of the colorant—included one or more amino-functionalized silicone polymers (F-1) in a total amount of 0.1 to 8.0 wt. %, preferably 0.2 to 5.0 wt. %, more preferably 0.3 to 3.0 wt. % and most preferably 0.4 to 2.5 wt. %.

Within the scope of a further particularly preferred embodiment, the colorant (F) comprises—based on the total weight of the colorant—one or more amino-functionalized silicone polymers (F-1) in a total amount of from 0.1 to 8.0 wt. %, preferably from 0.2 to 5.0 wt. %, more preferably from 0.3 to 3.0 wt. % and very particularly preferably from 0.4 to 2.5 wt. %.

### Pigments (F-2) in the Colorant (F)

As a second constituent essential to the present disclosure, the colorant (F) used in the process as contemplated herein comprises at least one pigment.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, the colorant (F) is comprises at least one colorant compound (F-2) chosen from the group of inorganic and/or organic pigments.

In a preferred embodiment, the colorant (F) comprises at least one inorganic and/or organic pigment (F-2).

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

As contemplated herein, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, the colorant (F) comprises at least one colorant compound (F-2) chosen from the group of inorganic pigments, which is preferably selected from the group of colored metal oxides, metal hydroxides, metal oxide hydrates, silicates, metal sulfides, complex metal cyanides, metal sulfates, bronze pigments and/or from colored mica- or mica-based pigments coated with at least one metal oxide and/or a metal oxychloride.

In a further preferred embodiment, the colorant comprises (a) at least one colorant compound (F-2) chosen from the group of pigments selected from mica- or mica-based pigments which are reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

Unipure Yellow LC 182 EM, Sensient, CI 77492 (Iron Oxides), Silica

In a further embodiment, the composition as contemplated herein may also comprise (a) one or more colorant compounds (F-2) selected from the group of organic pigments

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, the colorant (F) comprises at least one colorant compound (F-2) chosen from the group of organic pigments which is preferably selected from the group of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above mentioned conditions. The particles can, for example, be inorganic substrates, which can be aluminum, silica, calcium borosilate, calcium aluminum borosilicate or even aluminum.

For example, alizarin color varnish can be used.

Due to their excellent light and temperature resistance, the use of the above pigments in the colorant (F) of the process as contemplated herein is particularly preferred. It is also preferred if the pigments used have a certain particle size. As contemplated herein, it is therefore advantageous if the at least one pigment has an average particle size D₅₀ of 1.0 to 50 μm, preferably 5.0 to 45 μm, preferably 10 to 40 μm, 14 to 30 μm. The average particle size D50, for example, can be determined using dynamic light scattering (DLS).

The pigment(s) (F-2) constitute the second essential component of the colorant as contemplated herein and are preferably used in the agent in certain ranges of amounts. Particularly satisfactory results were obtained when the colorant included—based on the total weight of the colorant—one or more pigments (F-2) in a total amount of 0.01 to 10.0 wt. %, preferably 0.1 to 5.0 wt. %, more preferably 0.2 to 2.5 wt. % and most preferably 0.25 to 1.5 wt. %.

In another very particularly preferred embodiment, the colorant (F) comprises —based on the total weight of the colorant—one or more pigments (F-2) in a total amount of from 0.01 to 10.0 wt. %, preferably from 0.1 to 5.0 wt. %, more preferably from 0.2 to 2.5 wt. % and very particularly preferably from 0.25 to 1.5 wt. %.

As optional components, the colorants (F) used in the process as contemplated herein may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments.

Preferably, the direct dyes within the meaning of the present disclosure have a solubility in water (760 mmHg) at 25° C. of more than 1.0 g/L.

Direct dyes can be divided into anionic, cationic and non-ionic direct dyes.

In a further embodiment, the colorant (F) additionally comprises at least one anionic, nonionic and cationic direct dyes.

Suitable cationic direct dyes include Basic Blue 7, Basic Blue 26, HC Blue 16, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76.

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

In the course of the work leading to the present disclosure, it has been found that dyeings with good color intensities and fastness properties can also be produced with agents (F) comprising at least one anionic direct dye (F-2).

In a further embodiment, the colorant (F) comprises at least one anionic direct dye.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—COO—, —SO₃ present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Inventive acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

Acid dyes as contemplated herein have an ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In another embodiment, the colorant (F) comprises at least one anionic direct dye selected from the group of the nitrophenylenediamines, the nitroaminophenols, the azo dyes, the anthraquinone dyes, the triarylmethane dyes, the xanthene dyes the rhodamine dyes, the oxazine dyes and/or the indophenol dyes, the dyes from the abovementioned group each having at least one carboxylic acid group (—COOH), a sodium carboxylate group (—COONa), a potassium carboxylate group (—COOK), a sulfonic acid group (—SO₃H), a sodium sulfonate group (—SO₃Na) and/or a potassium sulfonate group (—SO₃K).

Suitable acid dyes may include, for example, one or more compounds selected from the following group: Acid Yellow 1 (D&C Yellow 7, Citronin A, Ext. D&C Yellow No. 7, Japan Yellow 403,CI 10316, COLIPA n° B001), Acid Yellow 3 (COLIPA n°: C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C.015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1;CI 20170;KATSU201;nosodiumsalt;Brown No. 201;RESORCIN BROWN;ACID ORANGE 24;Japan Brown 201;D & C Brown No. 1), Acid Red 14 (C.I.14720), Acid Red 18 (E124, Red 18; CI 16255), Acid Red 27 (E 123, CI 16185, C-Rot 46, Real red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodofluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA n° C.53, CI 45410), Acid Red 95 (CI 45425, Erythtosine,Simacid Erythrosine Y), Acid Red 184 (CI 15685), Acid Red 195, Acid Violet 43 (Jarocol Violet 43, Ext. D&C Violet n° 2, C.I. 60730, COLIPA n° C.063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA n° B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved radicals, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).

Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has an extremely high water solubility of more than 20 wt. %.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a solubility in water of more than 20 wt. % (25° C.).

In a further embodiment, the colorant (F) comprises at least one direct dye (F-2) selected from the group of acid yellow 1, acid yellow 3, acid yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct dye(s) can be used in different amounts in the colorant (F) depending on the desired color intensity. Particularly satisfactory results could be obtained if the colorant (F) —based on the total weight of the colorant (F)—comprises one or more direct dyes (b) in a total amount of from 0.01 to 10.0 wt. %, preferably from 0.1 to 8.0 wt. %, more preferably from 0.2 to 6.0 wt. % and very particularly preferably from 0.5 to 4.5 wt. %.

Furthermore, the colorant (F) may also contain at least one photochromic or thermochromic dye as the colorant compound (F-2).

Photochromic dyes are dyes that react to irradiation with UV light (sunlight or black light) with a reversible change in hue. In the process, the UV light changes the chemical structure of the dyes and thus their absorption behavior (Photochromie).

Thermochromic dyes are dyes that react to temperature changes with a reversible change in hue. In this process, the change in temperature alters the chemical structure of the dyes and thus their absorption behavior (Thermochromism).

The colorant (F) may contain—based on the total weight of the colorant (F)—one or more photochromic dyes (b) in a total amount of from 0.01 to 10.0 wt. %, preferably from 0.1 to 8.0 wt. %, more preferably from 0.2 to 6.0 wt. % and most preferably from 0.5 to 4.5 wt. %

The colorant (F) may contain—based on the total weight of the colorant (F)—one or more thermochromic dyes (b) in a total amount of from 0.01 to 10.0 wt. %, preferably from 0.1 to 8.0 wt. %, more preferably from 0.2 to 6.0 wt. %, and most preferably from 0.5 to 4.5 wt. % of

Further Optional Ingredients in the Agents (V) and/or (F)

In addition to the ingredients essential to the present disclosure already described, the pretreatment agent (V) and/or the colorant (F) may also contain other optional ingredients.

The agents may also contain other active ingredients, auxiliaries and additives, such as solvents; fatty ingredients such as C₈-C₃₀ fatty alcohols, C₈-C₃₀ fatty acid triglycerides, C₈-C₃₀ fatty acid monoglycerides, C₈-C₃₀ fatty acid diglycerides and/or hydrocarbons; polymers; structurants such as glucose, maleic acid and lactic acid, hair conditioning compounds such as phospholipids, for example lecitin and kephalins; perfume oils, dimethyl isosorbide and cyclodextrins; fiber structure-improving active ingredients, in particular mono-, di- and oligosaccharides such as glucose, galactose, fructose, fructose and lactose; dyes for coloring the product; anti-dandruff active ingredients such as piroctone olamine, zinc omadine and climbazole; amino acids and oligopeptides; protein hydrolysates on an animal and/or vegetable basis, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; Polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; Fats and waxes such as fatty alcohols, beeswax, montan wax and; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescing agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. Regarding other optional components and the quantities of these components used, explicit reference is made to the relevant manuals known to the specialist. The additional active ingredients and auxiliary substances are preferably used in the preparations as contemplated herein in quantities of 0.0001 to 25 wt. % each, 0.0005 to 15 wt. %, based on the total weight of the respective agent.

However, the purpose of the pre-treatment agent (V) is to remove all substances that have been deposited on the surface of the keratin material because of a previous application of a conditioner or styling agent. It is assumed that polymers and/or cationic surfactants can have a negative influence on a pigment colorant applied later due to their high affinity to the keratin material.

For this reason, it is particularly preferred not to use polymers and cationic surfactants in the pretreatment agent (V) as contemplated herein, or to minimize their amounts used.

In a further preferred embodiment, the total content of all polymers included in the pretreatment agent (V)—based on the total weight of the pretreatment agent—is below 0.5 wt. %, preferably below 0.1 wt. % and very preferably below 0.01 wt. %.

In the context of a further preferred embodiment, the total content of all cationic surfactants included in the pretreatment agent (V)—based on the total weight of the pretreatment agent—is below 0.5 wt. %, preferably below 0.1 wt. % and very preferably below 0.01 wt. %.

pH Value of Pretreatment Agent (V) and/or Colorant (F)

The pH values of the agents (V) and (F) as contemplated herein can be adjusted to a slightly acidic to alkaline pH.

In one embodiment, the pretreatment agent (V) has a pH of from 2.0 to 6.5, preferably from 2.5 to 6.0, more preferably from 3.0 to 5.5, and most preferably from 3.5 to 5.0. If pretreatment agents (V) with these pH values were used in the process as contemplated herein, colorations with particularly high color intensity could be obtained.

In another embodiment, the pretreatment agent (V) has a pH of from 7.0 to 11.5, preferably from 7.5 to 11.0, and most preferably from 8.0 to 10.5. If pretreatment agents (V) with these pH values were used in the process as contemplated herein, colorations with particularly good uniformity could be obtained.

The pH values of the colorant (F) as contemplated herein can be adjusted to a slightly acidic to alkaline pH. Very preferably, colorant (F) has a pH in the range from 5.0 to 10.0, preferably from 6.0 to 9.5, more preferably from 6.0 to 8.7, and most preferably from 6.0 to 7.5.

Alkalizing agents and acidifying agents known to those skilled in the art can be used to set the respective desired pH values. The pH values for the purposes of the present disclosure are pH values measured at a temperature of 22° C.

As alkalizing agents, the agents may contain, for example, ammonia, alkanolamines and/or basic amino acids.

The alkanolamines which can be used in the agent of the present disclosure are preferably selected from primary amines having a C₂-C₆ alkyl base which carries at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

Alkanolamines particularly preferred as contemplated herein are selected from 2-aminoethan-1-ol and/or 2-amino-2-methylpropan-1-ol. in a particularly preferred embodiment, the agent as contemplated herein comprises an alkanolamine selected from 2-aminoethan-1-ol and/or 2-amino-2-methylpropan-1-ol as alkalizing agent.

For the purposes of the present disclosure, an amino acid is an organic compound comprising at least one protonatable amino group and at least one —COOH or —SO₃H group in its structure. Preferred amino acids are amino carboxylic acids, especially α-(alpha)-amino carboxylic acids and ω-amino carboxylic acids, whereby α-amino carboxylic acids are particularly preferred.

As contemplated herein, basic amino acids are those amino acids which have an isoelectric point pI of greater than 7.0.

Basic α-amino carboxylic acids contain at least one asymmetric carbon atom. In the context of the present disclosure, both enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, the alkalizing agent is a basic amino acid from the group arginine, lysine, ornithine and/or histidine.

In addition, the product may contain other alkalizing agents, especially inorganic alkalizing agents. Inorganic alkalizing agents usable as contemplated herein are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Also, as contemplated herein, the desired pH value can be adjusted by employing a buffer system. A buffer or buffer system is usually understood to be a mixture of a weak or medium-strength acid (e.g., acetic acid) with a completely dissociated neutral salt of the same acid (e.g., sodium acetate). If some base or acid is added, the pH value hardly changes (buffering). The effect of the buffer substances included in a buffer solution is based on the scavenging reaction of hydrogen or hydroxide ions with the formation of weak acids or bases due to their dissociation equilibrium.

A buffer system can be formed from a mixture of an inorganic or organic acid and a corresponding salt of that acid.

Acids can be buffered by all salts of weak acids and strong bases, bases by salts of strong acids and weak bases. The strong hydrochloric acid (completely dissociated into ions) can be buffered, for example, by adding sodium acetate. According to the balance

H₃C—COONa+HCl [symbol] NaCl+H₃C—COOH

hydrochloric acid is converted by sodium acetate to the weak acetic acid with the formation of sodium chloride, which dissociates only to a small extent in the presence of an excess of sodium acetate. Buffers that are effective against both acids and bases are mixtures of weak acids and their salts.

Examples of buffer systems known from the literature are acetic acid/sodium acetate, boric acid/sodium borate, phosphoric acid/sodium phosphate and hydrogen carbonate/soda. The pH of the agent as contemplated herein can be adjusted, for example, by adding an inorganic or organic buffer system. For the purposes of the present disclosure, an inorganic buffer system is understood to be a mixture of an inorganic acid and its conjugate corresponding inorganic base.

For the purposes of the present disclosure, an organic buffer system is understood to be a mixture of an organic acid and its conjugate corresponding base. Due to the organic acid radical, the conjugate corresponding base of the organic acid is also organic. Here, the cation present to neutralize the charge of the acid anion can be inorganic or organic.

Examples of inorganic acids are sulfuric acid, hydrochloric acid and phosphoric acid (H₃PO₄). Phosphoric acid is a medium-strength acid that is particularly preferred.

A particularly well-suited inorganic acid is potassium dihydrogen phosphate Potassium dihydrogen phosphate has the molecular formula KH₂PO₄ and carries the CAS number 7778-77-0. Potassium dihydrogen phosphate has a molar mass of 136.09 g/mol. It is highly soluble in water (222 g/l at 20° C.) and reacts acidically in water. A 5% solution of potassium dihydrogen phosphate in water has a pH value of 4.4.

Another particularly suitable inorganic acid is sodium dihydrogen phosphate. Sodium dihydrogen phosphate has the molecular formula NaH₂PO₄ and carries the CAS numbers 7558-80-7 (anhydrate), 10049-21-5 (monohydrate) and 13472-35-0 (dihydrate). The anhydrous sodium dihydrogen phosphate has a molar mass of 119.98 g/mol. Sodium dihydrogen phosphate reacts acidically in aqueous solution.

Particularly preferred as a corresponding salt of the above two acids is dipotassium hydrogen phosphate. Dipotassium hydrogen phosphate has the molecular formula K₂HPO₄ and carries the CAS numbers 7758-11-4 (anhydrous) and 16788-57-1 (trihydrate). The anhydrous dipotassium hydrogen phosphate has a molar mass of 174.18 g/mol. Dipotassium hydrogen phosphate reacts alkaline in aqueous solution.

Also particularly preferred as a corresponding salt of the above two acids is disodium hydrogen phosphate. Disodium hydrogen phosphate has the molecular formula Na₂HPO₄ and carries the CAS numbers 7558-79-4 (anhydrous), 10028-24-7 (dihydrate), 7782-85-6 (heptahydrate) and 10039-32-4 (dodecahydrate). Anhydrous disodium hydrogen phosphate has a molar mass of 141.96 g/mol. Disodium hydrogen phosphate reacts alkaline in aqueous solution.

Examples of organic acids are citric acid, succinic acid, tartaric acid, lactic acid, acetic acid, malic acid, malonic acid and maleic acid.

Examples of the corresponding salts of these organic acids are the sodium and potassium salts of citric acid, the sodium and potassium salts of succinic acid, the sodium and potassium salts of tartaric acid, sodium and potassium salts of lactic acid, sodium and potassium salts of acetic acid, sodium and potassium salts of malic acid, sodium and potassium salts of malonic acid and sodium and potassium salts of maleic acid.

### Sequence of the Process Steps

As previously described, the pretreatment agent (V) is applied before the application of the colorant (F). In this context, it has been found to be particularly preferable to apply the pretreatment agent (V) to the keratin material, allow it to act for a certain period and then rinse it out again with water.

Thus, another subject matter is a process for coloring keratinous fibers, in particular human hair, comprising the following steps:

(1) Applying a pretreatment agent (V) to the keratinous fibers, the pretreatment agent (V) having been disclosed in detail in the description of the first subject matter of the present disclosure,
(2) Exposure of the pretreatment agent applied in step (1) to the keratinous fibers,
(3) Rinse out the pretreatment agent with water,
(4) Applying a colorant (F) to the keratinous fibers, the colorant having been disclosed in detail in the description of the first subject matter of the present disclosure,
(5) Exposing the keratinous fibers to the colorant applied in step (4), and
(6) Rinse out the dye with water.

Explicitly quite particularly preferred is a process for coloring keratinous fibers, in particular human hair, comprising the following steps in the order indicated:

(1) Applying a pretreatment agent (V) to the keratinous fibers, the pretreatment agent (V) having been disclosed in detail in the description of the first subject matter of the present disclosure,
(2) Exposure of the pretreatment agent applied in step (1) to the keratinous fibers,
(3) Rinse out the pretreatment agent with water,
(4) Applying a colorant (F) to the keratinous fibers, the colorant having been disclosed in detail in the description of the first subject matter of the present disclosure,
(5) Exposing the keratinous fibers to the colorant applied in step (4), and
(6) Rinse out the dye with water.

In step (1) of the process as contemplated herein, a pretreatment agent (V) comprising at least one anionic surfactant in a water-comprising carrier is applied to the hair.

In the following step, the previously applied pretreatment agent (V) is allowed to act on the keratin fibers. In this context, different exposure times of, for example, 30 seconds to 60 minutes are conceivable. Exposure times of 30 seconds to 5 minutes are preferred.

Following the action of the pretreatment agent (V) on the keratin fibers, it is finally rinsed with water in step (3). Here, in a preferred embodiment, the pretreatment agent (V) is washed out with water only, i.e., without the aid of an aftertreatment agent or shampoo not as contemplated herein.

In principle, the user is now free to choose the period between the application of the two agents (V) and (F).

However, the coloring agent (F) must be applied to keratin material or hair that is in a condition as it is after rinsing off the pretreatment agent (V). For this reason, it is essential to the present disclosure that no other agents, such as other conditioners, styling agents are applied between the application of the two agents (V) and (F). In this way, the maximum time interval between the application of the two agents (V) and (F) is usually limited to a maximum of 24 hours.

It has been found to be preferable if between the rinsing of the pretreatment agent (V) with water and the application of a coloring agent (F) to the keratinous fibers there is a period of not more than 24 hours, preferably not more than 12 hours, further preferably not more than 6 hours and very particularly preferably not more than 3 hours.

In the context of a further preferred embodiment, between steps (3) and (4) there is a period of at most 24 hours, preferably of at most 12 hours, further preferably of at most 6 hours and very particularly preferably of at most 3 hours.

In the context of a further preferred embodiment, step (4) takes place directly after step (3). Step (4) is used to apply the colorant.

The exposure of the colorant (F) to the keratinous fibers in step (5) may be for a period ranging from 15 seconds to 30 minutes, for example, preferably for a period ranging from 30 seconds to 15 minutes, more preferably for a period ranging from 1 to 15 minutes.

After that, the colorant (F) is finally rinsed with water in step (6). Here, in a preferred embodiment, the colorant (F) is washed out with water only, i.e., without the aid of an aftertreatment agent or shampoo not as contemplated herein.

### Multi-Component Packaging Unit

To increase user convenience, the user is preferably provided with all the necessary agents in the form of a multi-component packaging unit (kit-of-parts).

Thus, another object of the present disclosure is a multi-component packaging unit (kit-of-parts) for dyeing keratinous material, comprising separately prepared

a first container comprising a pretreatment agent (V), the pretreatment agent (V) having been disclosed in detail in the description of the first subject matter of the present disclosure, and
a second container comprising a colorant (F), the colorant (F) having been disclosed in detail in the description of the first subject matter of the present disclosure,

Concerning the further preferred embodiments of the multicomponent packaging unit as contemplated herein, mutatis mutandis what has been said about the processes as contemplated herein applies.

### EXAMPLES

### 1. Formulations

The following formulations were prepared (all data in wt. % unless otherwise stated):

| Conditioner | (C) |
|---|---|
| Isopropyl myristate | 0.8 |
| Cetearyl alcohol | 3.4 |
| [3-(Behenoyloxy)-2-hydroxypropyl]trimethylammonium chloride | 1.0 |
| (INCI: BEHENOYL PG-TRIMONIUM CHLORIDE) | |
| Behentrimonium chloride | 0.5 |
| Citric acid | 0.5 |
| Methyl paraben, Na salt | 0.3 |
| Glycerin | 0.2 |
| Cosmedia Triple C (BASF, Polyquaternium-37, Dicaprylyl | 0.2 |
| Carbonate, Lauryl Glucoside) | |
| Polyvinylpyrrolidone/vinyl acetate copolymer (60/40) | 1.0 |
| Phenxoy ethanol | 0.4 |
| Perfume | 0.4 |
| Water | ad 100 |

| Pretreatment agent (V) | (V1) | (V2) |
|---|---|---|
| Texapon K 12 G Ecocert | — | 10.0 |
| Sodium lauryl sulfate (BASF, CAS No. | | |
| 151-21-3) | | |
| Sodium laureth sulfate (sodium lauryl ether | 8.5 | — |
| sulfate, ethoxylated with 2 EO) CAS No. | | |
| 68891-38-3 | | |
| Cocoamidopropylbetaine | 1.5 | — |
| Ammonia/citric acid | ad pH 4.6 | ad pH 10.0 |
| Water | ad 100 | ad 100 |

| Coloring agent (F) | F |
|---|---|
| Cetyl alcohol | 3.0 g |
| Stearyl alcohol | 3.0 g |
| Ceteareth-30, (Cetearyl alcohol, ethoxylated 30 EO) | 1.5 g |
| Potassium dihydrogen phosphate | 0.07 |
| Disodium hydrogen phosphate | 0.15 |
| Unipure Red LC3071, organic pigment CI 15850 | 1.0 g |
| 1.2-propanediol | 10.0 g |
| Dow Coming 2-8566 (Siloxanes and Silicones, 3-[(2- | 1.0 g |
| Aminoethyl)amino]-2-methylpropyl Me, Di-Me-Siloxane“ | |
| Water | ad 100 |

### 2. Application on Strands

First, hair strands (Kerling company) were treated with the conditioner. For this purpose, the conditioner (0.2 g per g of hair) was applied to the moistened hair, left to act for a period of 5 minutes and then rinsed with water.

The respective pretreatment agent (V) was applied to the still wet strands. For this purpose, 0.2 g of pretreatment agent (V) per g of hair was applied to each strand, massaged in for 30 seconds and then rinsed with water.

A reference strand was treated directly with the colorant (F) without applying the pretreatment agent (V).

The coloring agent (F) was applied to the still damp hair directly afterwards. For this purpose, 0.2 g of colorant (F) per g of hair strand was massaged in, left to act for 5 minutes and then rinsed out again with water.

After completion of the treatment, the hair strands were calorimetrically measured using a Data color Spectraflash 450 colorimeter.

The dE value used to evaluate the distinct color intensities is derived from the L*a*b* colorimetric values measured on the respective strand part as follows:

dE=[(Lᵢ−L₀)²+(aᵢ−a₀)²+(bᵢ−b₀)]^{1/2}

L₀, a₀ and b₀=Measured values of the reference strand

Lᵢ, aᵢ and bᵢ=Measured values of the coloration obtained with the process as contemplated

herein

| For | | Pretreatment | Dyeing | | | | |
|---|---|---|---|---|---|---|---|
| e.g.: | Conditioner | agent | agents | L | a | b | dE |
| 1 | (C) | — | (F) | 31.70 | 30.44 | 11.41 | — |
| 2 | (C) | (V1) | (F) | 21.96 | 31.02 | 11.73 | 9.8 |
| 3 | (C) | (V2) | (F) | 26.24 | 32.30 | 10.06 | 5.9 |

### 3. Application in Hair Studio

In 3 subjects, the conditioner was applied first. For this purpose, the conditioner was applied to damp hair, left to act for a period of 5 minutes and then rinsed with water.

Then, one of each of the two pretreatment agents (V1) and (V2) were applied to the still damp hair of two subjects, massaged in for 30 seconds, and then rinsed out with water.

No pretreatment agent (V) was applied to any subject.

Then, each subject was treated with the stain (F). For this purpose, the dye (F) was massaged into the hair for 5 minutes and then rinsed again with water.

The intensity and uniformity of the coloration were visually assessed by trained individuals.

| test | | Pretreatment | Dyeing | Color | |
|---|---|---|---|---|---|
| person | Conditioner | agent | agents | Intensity | Uniformity |
| 1 | (C) | — | (F) | + | + |
| 2 | (C) | (V1) | (F) | +++ | ++ |
| 3 | (C) | (V2) | (F) | ++ | +++ |

+++ = very good
++ = medium
+ = poor

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A process for dyeing keratinous material comprising the following steps:
Applying a pretreatment agent (V) to the keratinous material, the pretreatment agent being disposed in a water-containing carrier, wherein the pretreatment agent (V comprises:
(V-1) at least one anionic surfactant, and
Applying a colorant (F) to the keratinous material, wherein the colorant comprises
(F-1) at least one amino-functionalized silicone polymer and
(F-2) at least one pigment.

2. The process according to claim 1, wherein the pretreatment agent (V) comprises
(V-1) at least one anionic surfactant of the formula (T-1), 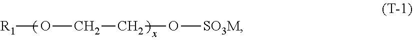
where
R₁ represents a linear or branched, saturated or unsaturated C₈-C₃₀ alkyl group,
x stands for an integer from 0 to 50, and
M is a hydrogen atom, ammonium (NH₄)+ or an equivalent of a monovalent or polyvalent cation.

3. The process according to claim 2, wherein the pretreatment agent (V) comprises
at least one anionic surfactant (V-1) of formula (T-1), wherein
R₁ is a linear, saturated or unsaturated C₁₂-C₁₈ alkyl group,
x is an integer from 0 to 5,
M represents a hydrogen atom or an alkali metal cation.

4. The process according to claim 1, wherein the pretreatment agent (V) comprises—based on the total weight of the pretreatment agent the at least one anionic surfactant(s) (V-1) in a total amount of from 2.0 to 18.0 wt. %.

5. The process according to a claim 1, wherein the pretreatment agent (V) further comprises (V-2) at least one zwitterionic and/or amphoteric surfactant.

6. The process according to claim 5, wherein the pretreatment agent (V) comprises
(V-2) at least one zwitterionic surfactant selected from the surfactants of the formula (T-2), (T-3), (T-4) and/or (T-5), 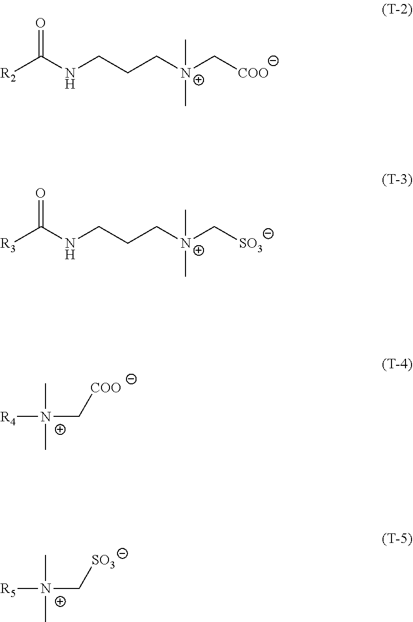
where
R2, R3, R4, R5 independently represent a linear or branched, saturated or unsaturated C₈-C₃₀ alkyl group, preferably.

7. The process according to claim 1, wherein the pretreatment agent (V) further comprises—based on the total weight of the pretreatment agent—one or more zwitterionic and/or amphoteric surfactants (V-2) in a total amount of 0.5 to 8.5 wt. %.

8. The process according to claim 1, wherein the at least one amino-functionalized silicone polymer (F-1) comprises at least one secondary amino group.

9. The process according to claim 1, wherein the at least one amino-functionalized silicone polymer (F-1) comprises at least one structural unit of the formula (Si amino), 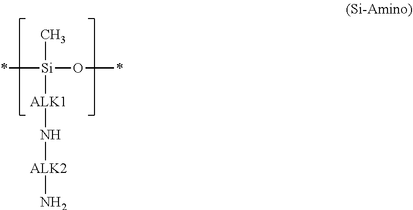
where
ALK1 and ALK2 independently represent a linear or branched C₁-C₂₀ divalent alkylene group.

10. The process according to claim 1, wherein the cm at least one amino-functionalized silicone polymer (F-1) comprises structural units of the formula (Si-I) and of the formula (Si-II)

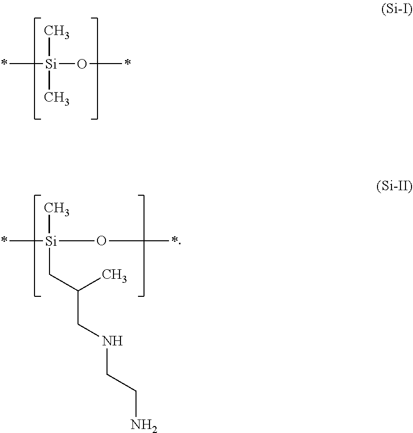

11. The process according to claim 1, wherein the colorant (F) comprises—based on the total weight of the colorant—the at least one amino-functionalized silicone polymer(s) (F-1) in a total amount of from 0.1 to 8.0 wt. %.

12. The process according to claim 1, wherein the at least one pigment (F-2) is selected from the group of colored metal oxides, metal hydroxides, metal oxide hydrates, silicates, metal sulfides, complex metal cyanides, metal sulfates, bronze pigments and/or from mica- or mica-based colored pigments coated with at least one metal oxide and/or a metal oxychloride.

13. The process according to claim 1, wherein the at least one pigment (F-2) is selected from the group of carmine, quinacridone, phthalocyanine, Sorgho, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

14. The process according to any one of claims 1 to 13, characterized in that the colorant (F) contains—based on the total weight of the colorant—one or more pigments (F-2) in a total amount of from 0.01 to 10.0 wt. %, preferably from 0.1 to 5.0 wt. %, more preferably from 0.2 to 2.5% by weight and very particularly preferably from 0.25 to 1.5 wt. %.

15. A process for coloring keratinous fibers comprising the following steps:
(1) Applying the pretreatment agent (V) as set forth in claim 1 to the keratinous fibers,
(2) Exposing the pretreatment agent applied in step (1) to the keratinous fibers,
(3) Rinsing the pretreatment agent from the keratinous fibers with water,
(4) Applying the colorant (F) as set forth in claim 1 to the keratinous fibers,
(5) Exposing the keratinous fibers to the colorant applied in step (4), and
(6) Rinsing the colorant (F) from the keratinous fibers with water.

16. The process according to claim 15, wherein between steps (3) and (4) there is a period of at most 24 hours.

17. Kit-of-parts for dyeing keratinous material, comprising separately packaged
a first container comprising a pretreatment agent (V) as set forth in claim 1,
a second container comprising a colorant (F) as set forth in claim 1.

18. The process according to claim 1, wherein the pretreatment agent (V) comprises —based on the total weight of the pretreatment agent—the at least one anionic surfactant(s) (V-1) in a total amount of from 8.0 to 12.0 wt. %.

19. The process according to claim 5, wherein the pretreatment agent (V) comprises (V-2) at least one zwitterionic surfactant selected from the surfactants of the formula (T-2), (T-3), (T-4) and/or (T-5), 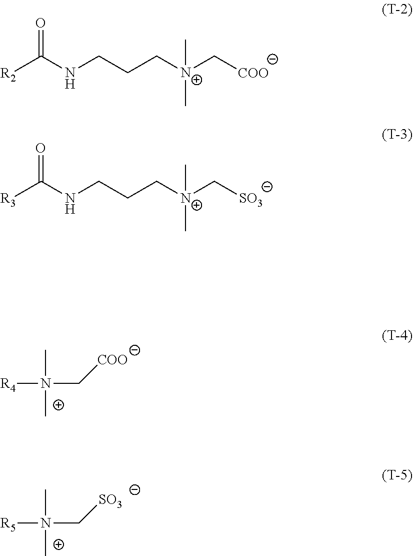
where
R2, R3, R4, R5 independently represent a linear or branched, saturated or unsaturated C₁₂-C₁₈ alkyl group.

20. The process according to claim 18, wherein the colorant (F) comprises—based on the total weight of the colorant—the at least one amino-functionalized silicone polymer(s) (F-i) in a total amount of from 0.4 to 2.5 wt. %.
